(19) United States
(12) Patent Application Publication
(10) Pub. No.: US 2003/0149996 A1
(43) Pub. Date: Aug. 7, 2003

(54) METHOD FOR PRODUCING POLYCLONAL AND MONOCLONAL ANTIBODIES

(76) Inventor: GEORG S. WENGLER, OBERHOFEN (AU)

Correspondence Address: KNOBBE MARTENS OLSON & BEAR LLP, 2040 MAIN STREET, FOURTEENTH FLOOR, IRVINE, CA, 92614, US

(21) Appl. No.: 09/242,856

(22) PCT Filed: Jun. 23, 1998
(86) PCT No.: PCT/EP98/03844

(30) Foreign Application Priority Data
Jun. 23, 1997 (DE) 197 26 597.9

(51) Int. Cl. A01K 67/00; A61K 48/00; C12N 5/06

(52) U.S. Cl. 800/8; 800/9; 800/13; 435/325; 435/326; 435/340; 435/346; 424/93.1; 424/93.3; 424/93.7

(57) ABSTRACT

This invention relates to a method for using an animal organism to produce monoclonal antibodies and/or human growth factors and/or for producing human immunoglobulins and blood components, and/or for producing polyclonal antiserums and/or for testing potential vaccines against infections. According to the invention, a human or animal stem cell is transplanted into a fertilised ovum of the animal organism to produce an intact human or animal haematopoiesis.

### DESCRIPTION

The present invention relates to a method for using an animal organism according to claim 1 and, in particular, to a method for producing human and mouse-monoclonal antibodies using at least one animal organism according to claim 10 and claim 15, respectively, and to a bioreactor according to claim 20.

Many strategies for diagnosing and treating human diseases are based on the results of animal tests. Various test models are here available to examine, for instance, questions and problems regarding pathogenesis, the sensitivity of diagnosing tests, the efficiency and reliability of therapeutic agents and vaccines.

Mice, rats and rabbits and then dogs, sheep, goats and Primates are the animals most often used in animal tests. The smaller the animal is, the lower are the costs for keeping the animal. Moreover, the breeding times are normally shorter for small animals. Highly developed techniques, e.g. transgenic systems, have mainly been developed for mice (see, for instance, DE 3301249 C2).

The drawback of tests in which mice are used is the great distance in the evolutionary origin of man and mouse. Therefore, many results which are achieved in tests using mice are just a reference point, but do not furnish evidence of an exact transferability to man.

In view of the evolutionary closeness of non-human Primates to man, there would be the best possibilities for animal models in this area. Restrictions, however, are due to the high costs and the discussions which especially arise in this area time and again and regard the ethical problems basically encountered in the case of animal tests.

Of course, such problems would in particular arise from tests on man though those would in principle be the most promising ones.

However, since modern medicine has made great progress in its diagnosing and treating methods because of the knowledge of many genetic diseases, there continues to be a great demand for providing suitable search models.

A new model for studying human diseases is based on the use of sheep into the fetus of which human stem cells are transplanted in utero (literature: Flake A W, Harrison M R, Adzick N S, Zanjani E D: Transplantation of Fetal Hematopoietic Stem Cells in Utero. The Creation of Hematopoietic Chimeras. Science 233: 766, 1986; Zanjani E, Almeida-Porada G. Flake A; Retention and Multilineage Expression of Human Hematopoietic Stem Cells in Human-Sheep Chimeras. Stem Cells 13: 101, 1995). So far, however, this model has only been used for studies regarding the investigation of the immunological immaturity of a fetus and the possibilities of producing tolerance to human xenografts.

It is the object of the present invention to provide a method for using an animal organism for producing monoclonal antibodies and/or for testing human growth factors and/or for producing human immunoglobutins and blood components, and/or for producing polyclonal antisera and/or for testing potential vaccines against infections, in particular HIV infections, which method provides relative simple test and production conditions of high efficiency and reproducibility.

This object is achieved by the features of claim 1.

Subclaims 2 to 9 relate to advantageous developments of said method.

In claim 10 a method for producing human monoclonal antibodies is defined as a particularly preferred embodiment of the present invention.

Apart from the above-explained fundamentals, the present invention is based on the following concept:

Claim 1 teaches the use of an animal organism for different production and test methods, the organism exhibiting tolerance to human cells by virtue of the transplantation of a human or animal stem cell into an inseminated ovum of the animal organism. This treatment is not concerned with the exchange of genetic material or with genetic manipulation.

The transplantation of tissue (cells, organs) faces two essential problems:

a. Host versus graft reaction (recipient/donor reaction):

The recipient, unless it is HLA (tissue type)-identical, rejects any foreign material, for instance donor cells or donor organs. In the following text everything that is foreign will be referred to as “not oneself”.

b. Graft versus host reaction (donor/recipient reaction):

The donor (above all lymphocytes) attacks tissue of the recipient. Every individual learns the meaning of “oneself” (i.e. “not foreign”) In an organ (the thymus) in the first period of his development (in mammals in the first period of gestation). Everything that lateron is not “oneself” is qualified as “not oneself” (i.e. “foreign”) (with a few exceptions).

However, it is possible with the introduction or transplantation of any type of cell into a fetus in the first third of the gestation period to suggest to the recipient that the transplanted (“foreign”) material is “oneself” because at that time the fetus as the recipient is not yet capable of identifying the material received as “not oneself”.

In such a case one talks about the previously mentioned “tolerance” to a tissue that is “not oneself”. With the tolerance achieved, it is later possible to transplant e.g. organs (such as heart, kidney, liver) from the same donor into the recipient without provoking any rejection reactions.

The following cells are suited as cells that can be transplanted according to claim 1:

Hematopoietic stem cells from bone marrow, umbilical cord, blood, fetal liver or spleen and from circulating blood stem cells in the peripheral blood circulation;

embryonal stem cells (every embryonal cell exhibits a certain omnipotence with respect to self-renewal and differentiation up to a certain stage);

any kind of cell culture (such as cell hybridoma) and tissue.

According to the invention any species is in principle suited as a recipient, i.e. as an animal organism. Particularly preferred are, however, sheep, goat, rabbit, cow and chicken.

Any species would in principle also be suited as a donor of the cells to be transferred, but preference is given to man, mouse, cell cultures, hybridomas and chicken.

Particularly preferred is the use of horses or sheep as animal organisms or recipients according to claim 1, for the anatomical situation of sheep during the gestation period permits a manipulation of the fetus under ECHO CONTROL already at an early time. In comparison with known methods (e.g. opening the abdominal cavity or uterus), the performance of an echo-controlled fetal manipulation with sheep (but also with rabbit or the egg of a chicken) is a minimal operation which hardly stresses the animal as the recipient. Hence, the present invention also takes into account the effort to spare the animal as much as possible while the method according to the invention is performed. Moreover, there is the advantage that a manipulation on the fetus affects the otherwise normal development and mortality of said fetus either little or not at all.

The rabbit should be mentioned as a further preferred recipient because its gestational physiology is considerably closer to the Primate than that of sheep. Furthermore, there is the advantage of a high reproductivity and a short gestation period.

The cow as a recipient should be mentioned as being particularly advantageous insofar as tests performed within the scope of the present invention have shown that the milk of cow, for instance when corresponding production steps are taken, contains high amounts of antibodies (immunoglobulins) which can very easily be isolated and further processed by withdrawing the milk.

The egg (e.g. of chicken to ostrich, resulting in different sizes, different hatching times and different costs) would be well suited for mass production, as the whole fetal development takes place outside the maternal body. Chicken eggs have a particularly short hatching time together with small costs and easy keeping. Monoclonal antibodies could be isolated from eggs laid at a later time, which have a high amount of immunoglobulins.

Apart from the above-explained steps of isolating or growing donor cells which are to be injected, and of transplanting said cells within the first third of the gestation period, preferably towards the end of the first third of gestation, intraperitoneally into the fetus (recipient), a further preferred step of the method which should be mentioned is the step that after the recipient has been born Its tolerant state is confirmed, preferably by molecular biological methods or with the so-called FACS (Fluorescence Activated Cell Sorting) method.

In summary, it should here be noted that the method according to the invention exploits the induced tolerance of the recipient (animal organism) to carry out a multitude of production and test methods. The results of the tests can be transferred to man in the case of the transplantation of human stem cells. It is here of particular advantage that the tolerance does not alter the fact that the organism to be used is an animal in which, for instance, through the transplantation of human hematopoietic stem cells in utero (IUT), a tolerance is produced through a resulting ratio of blood cells of the recipient (e.g. of sheep) to those of the donor (man) of about 90% to 10% (animal to human blood).

Under these conditions the animal organism can be used in the following way within the scope of the method or use according to the invention:

Testing the most different growth factors for human blood formation (such as Colony Stimulating Factor CSF: G-CSF, GM-CSF, and interleukins: IL-3, SCF, IL-6, IL-7, IL-12 . . . ). The special advantage would be that the animal with a human blood amount could directly be used for testing purposes without the need for further preparatory measures. Hence, it could e.g. be checked whether the human blood formation as a whole of 10% could be increased to a higher percentage or whether only a few lines, such as granulocytes or lymphocytes, can be increased.

Another possibility of using the animal organism according to claim 1 would consist in infecting human blood cells in said organism with pathogens, such as HIV, malaria or leishmanea. In such a case it would be possible to test new drugs and vaccines on a functioning human blood system which, however, is present in a living animal.

A further possibility of testing drugs for tumor or leukemia cells could also be tried out on the animal according to claim 1.

Furthermore, there is the possibility of producing human (or animal) blood products, such as immunoglobulins (which are produced by B cells) or interleukins or hematopoietic growth factors. The main problem regarding human blood products from donor blood, i.e. the risk of infections with HIV, HBV, HCV, etc., could advantageously be avoided according to the invention.

A further possibility according to the invention is the production of specific polyclonal immune sera (polyclonal immunoglobulins). For carrying out this inventive method or intended use, human blood cells are stimulated (vaccinated) in the tolerant animal organism with specific antigens, such as tetanus, rabies, etc., which causes a specific immune reaction with formation of polyclonal immunoglobulins. The same problems as are found in the previously explained application could thereby be eliminated because so far the said immunoglobulins have been used for a passive immunization after Infection without initial protection and had to be isolated from persons who suffered from said infection, namely from the sera of said persons.

A further basic application which is made possible by the method according to the invention is the mass production of mouse-monoconal antibodies. So far such antibodies have been isolated using mice, which, however, yields only very small quantities per animal.

With the method of the invention according to claim 15, it is possible to induce tolerance to mouse cells in another animal organism, such as sheep or horse, with the aim to considerably increase the amount of isolated mouse-monoclonal antibodies.

There are in principle three possibilities of inducing tolerance:

carrying out an in utero (intrauterine) transplantation of hybridoma cells, which produce monoclonal antibodies, directly into the embryo, e.g. sheep; however, there is a certain risk that the embryo will be overgrown by tumor;

an in utero transplantation of hybridoma cells which have been irradiated prior to injection and which produce monoclonal antibodies so that said hybridoma cells are no longer dividable, but are still tolerance-inducible.

inducing tolerance by transplantation of fetal liver from embryos of the Balb-C mouse inbred strain.

After transplantation and after birth of the animal (e.g. sheep) provided with tolerance by transplantation, mouse hybridoma cells can now be injected intraperitoneally without any foreign-body reaction being observed. The injected hybridoma cells multiply and start to form specific monoclonal antibodies. Clinically, this is e.g. visible from the formation of an ascites. The ascites contains hybridoma cells and serum fluid with the desired antibodies. These antibodies can be isolated from said fluid with the help of known methods. The amount of ascites and thus the amount of the antibodies to be isolated depends on the respective recipient, but lies e.g. within the liter range when sheep or similarly large animals are used, and is thus considerably higher than in methods which have so far been carried out with mice. Furthermore, monoclonal antibodies (mAK) can be isolated with the same isolating method from the milk of sheep which contains a high amount of immunoglobulins.

A further, particularly preferred application is the method according to claim 10. This method is a method for producing human monoclonal antibodies. Thanks to the induction of tolerance in the animal, in particular sheep or cow, to human cells by way of transplanting hematopoietic stem cells, the human immune system can be stimulated in the animal with any antigen (such as malaria, TBC, rabies, tumors, HIV, etc.) by subcutaneous injection. The stimulation has the effect that reactive B cells are formed which are isolated and fused with immortal myeloma cells. The resulting hybridomas can now be injected intraperitoneally into another animal which is tolerant to said myeloma cells. After injection the cells are growing and produce human antibodies which, e.g., can be removed from the resulting ascites or the milk or the resulting blood serum by suitable methods.

This method has considerable advantages over the methods which have so far been known and which in the production of monoclonal antibodies have been limited to mice, since the subsequent growth of hybridomas from mouse and human cells (which represent heterohybridomas, cf. the above-mentioned patent DE 3301249 C2) does not yet function in culture media.

Therefore, the inventive method according to claim 10 is of special advantage insofar as it permits the production of human monoclonal antibodies in which great hopes are placed with respect to the treatment of tumors. Since human monoclonal antibodies can be produced with the method of the invention, there are other great advantages over the presently produced mouse antibodies, since these only function well in vitro. However, when mouse-monoclonal antibodies (mouse mAb) are used in man, it often happens that rejection reactions take place since the mouse mAb (proteins) are recognized as “not oneself”. With the method of the invention, however, this problem could be avoided because human tolerance is first induced and the human immune system is then stimulated to form antibodies by the administration of tumor cells. Hence, it is possible to produce human monoclonal antibodies by using an animal organism.

Furthermore, the invention relates to a bioreactor according to claim 20 and the appended subclaims 21 to 26.

With the term “bioreactor” the present invention covers parts of animals which are kept alive with the aid of artificial life-sustaining equipment insofar as the body which is tolerant to a donor has a functioning blood circulation and a functioning organ in which monoclonal antibodies can be produced after the injection of hybridoma cells which cause an antibody reaction.

This definition, however, covers a naturally living mammal or a bird. In the case of mammals, particular preference is above all given to horses and sheep since particularly large amounts of antibodies can be produced in these animals.

Hence, with the definition “bioreactor”, the present invention covers both of the above-explained possibilities, with the use of artificially life-sustained animal body parts having the advantage that the animal as such is dead, so that it no longer feels anything, in particular no pain. However, it should be noted that, in particular, suffering caused by pain can be kept very low even in cases where naturally living animals are used.

It should here be clarified that the definition “bioreactor” covers both the mouse-tolerant and mouse/man-tolerant animal body part and also the mouse- or mouse/man-tolerant animal prior to injection of the hybridoma cells causing an antibody reaction.

Further details, features and advantages of the present invention will become apparent from the following description taken in conjunction with the drawing, in which:

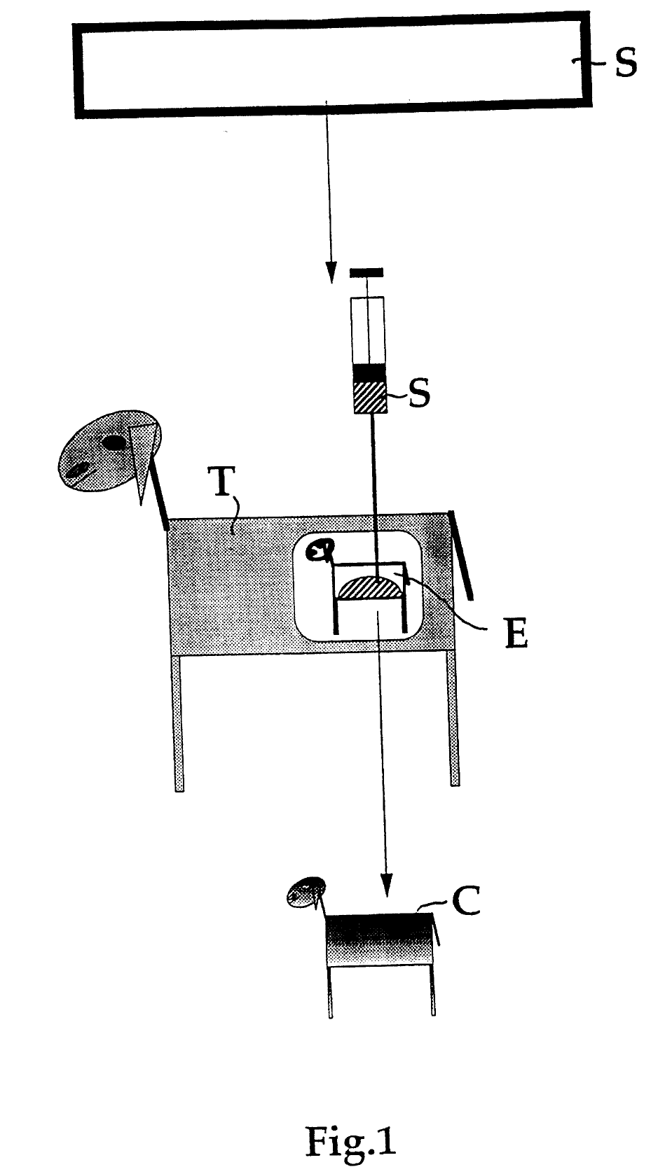

FIG. 1 shows the principle of inducing tolerance in an animal organism;

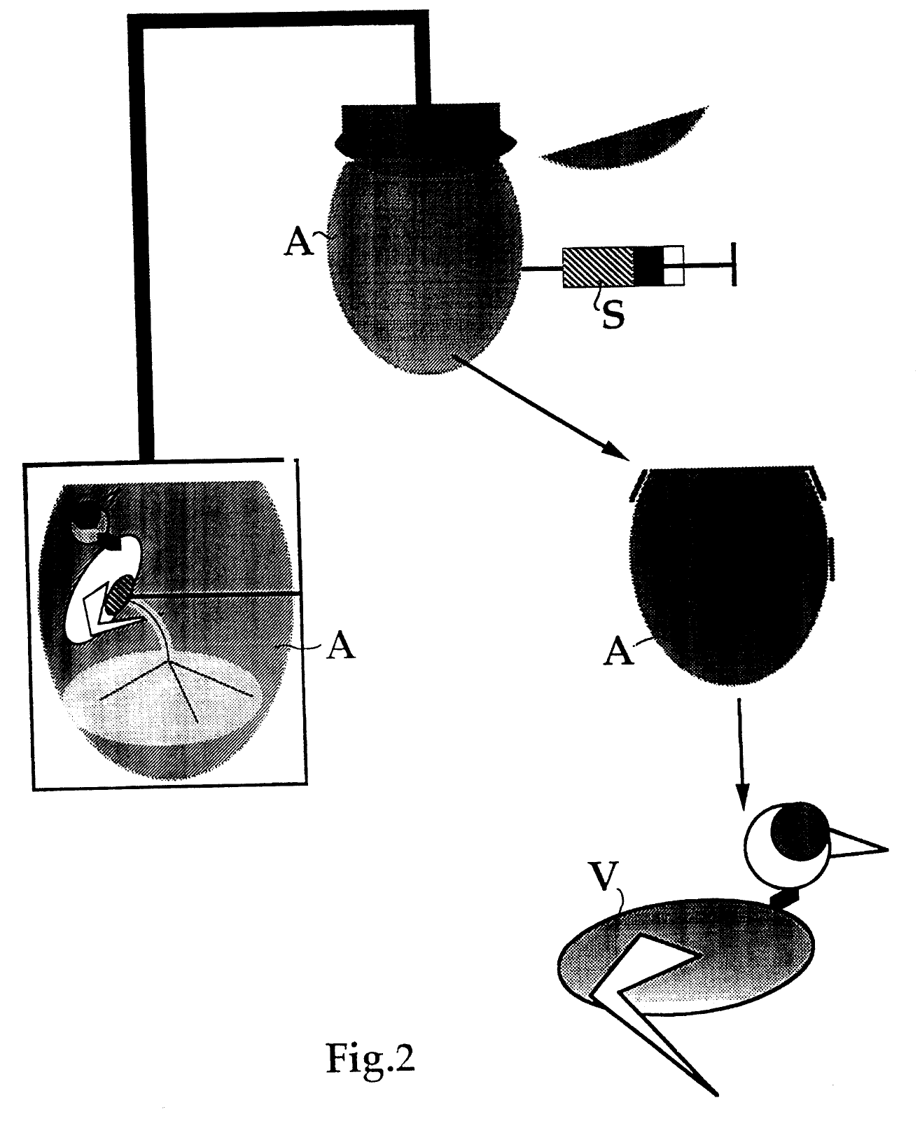

FIG. 2 is an illustration according to FIG. 1, showing a second possibility of inducing tolerance in an animal organism;

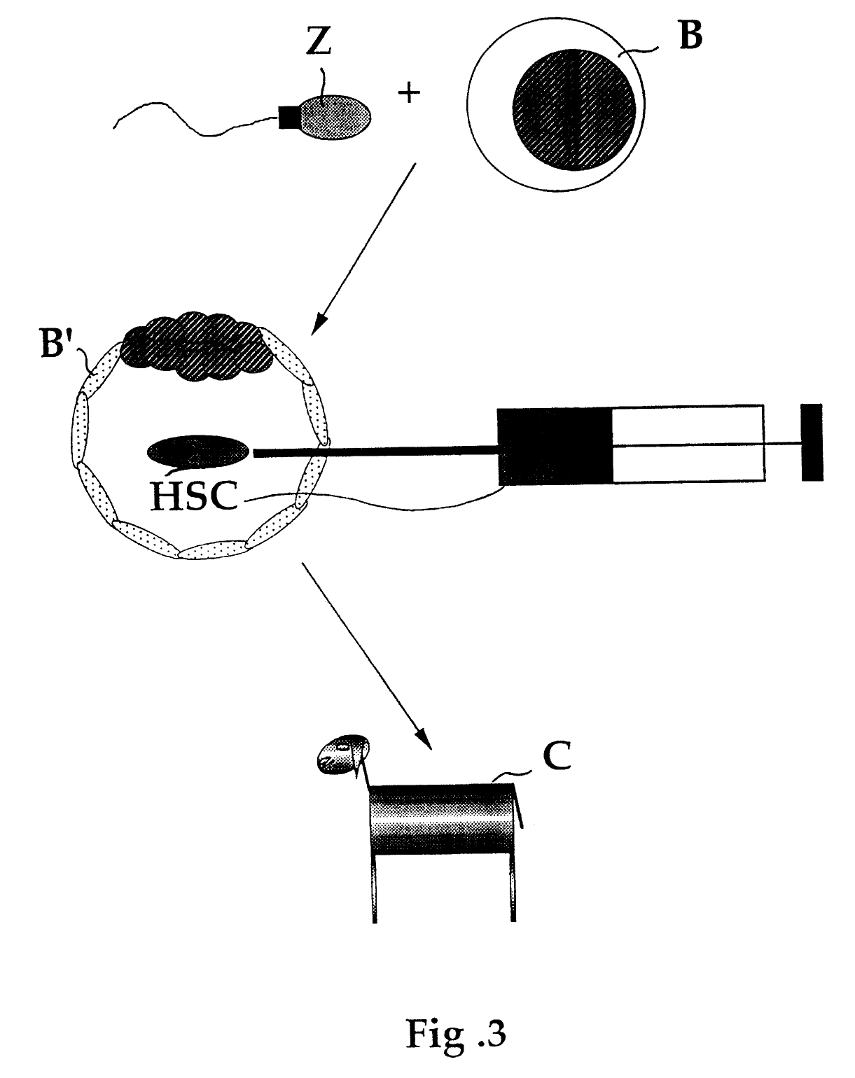

FIG. 3 is an Illustration according to FIGS. 1 and 2, showing a third possibility of inducing tolerance in an animal organism,

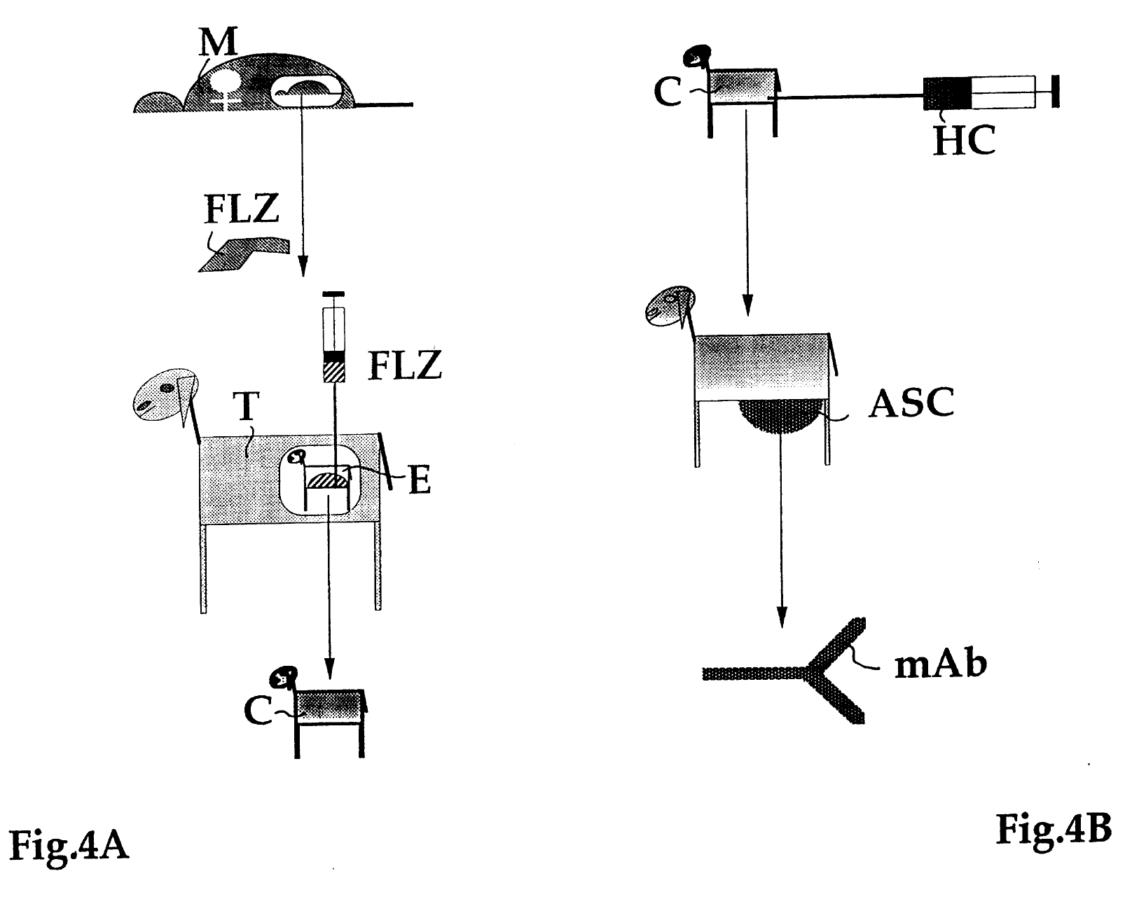

FIGS. 4A and 4B show a principle of how to produce mouse mAb; and

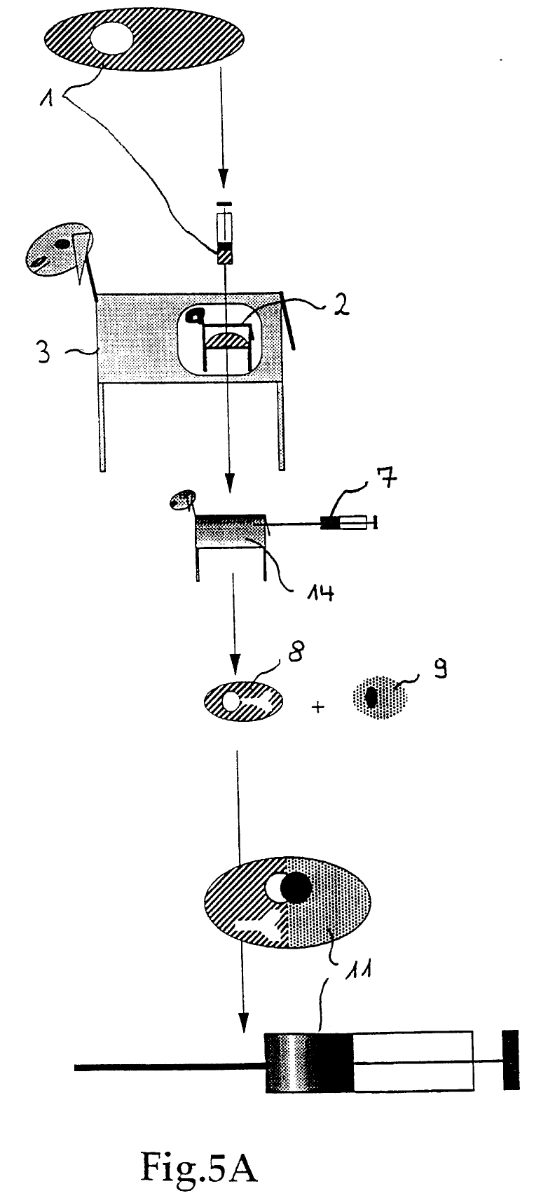

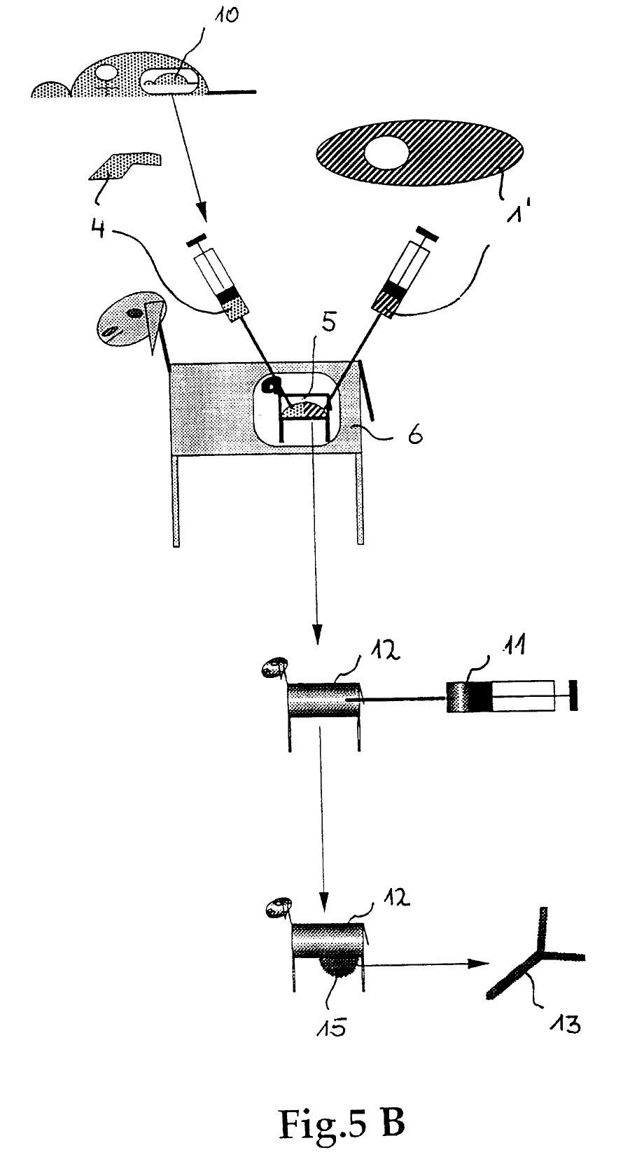

FIGS. 5A and 5B show a principle to explain a method of producing human monoclonal antibodies.

FIG. 1 is a schematic simplified view showing how tolerance can be induced by the in utero transplantation of donor cells S in an embryo E of a carrier animal T. As has been explained above, donor cells S are injected in the first third of the gestation period during which the foreign cells cannot be recognized yet by the embryo as foreign bodies. This results in an immune system which identifies the donor cells S injected in the first third as its own cells. Hence, birth is given to an animal organism C which exhibits the respective tolerance.

FIG. 2 illustrates a second possibility of producing an animal with a specific tolerance. In this case tolerance is induced in the egg of a bird (e g. chicken or ostrich). The egg A is here also provided with donor cells S and is subsequently hatched out. The result is an animal In the form of a bird V which exhibits the corresponding tolerance. The method is preferably carried out under ultrasonic control.

FIG. 3 illustrates an example of a basically possible in-vitro transplantation. To this end egg B is first of all artificially inseminated with a seminal cell Z. Foreign cells, such as hematopoietic stem cells HSC, are injected into the inseminated seminal cell B′. Subsequently, the inseminated egg which is provided with stem cells can be injected into the uterus of a carrier animal for the remaining gestation period. This, in turn, yields an animal C with a tolerance corresponding to the injected cells.

FIG. 4A and FIG. 4B diagrammatically show a method for producing monoclonal mouse antibodies.

The step of inducing tolerance is first explained in FIG. 4A. The donor is in this instance a mouse, normally a mouse M of the inbred strain Balb-C. To induce mouse tolerance in the embryo E of a carrier animal T (e.g. sheep), fetal liver cells FLZ may e.g. be Implanted or injected into the embryo in the first third of the gestation period. After birth there is an animal C with a mouse tolerance.

According to the method steps in FIG. 4B, this animal C is used for producing the monoclonal antibodies. To this end hybridoma cells HC of the donor (a mouse of the Balb-C inbred strain) is injected into the animal C. The result is the growth of antibody-producing cells, which becomes for example apparent from the development of an ascites ASC. The monoclonal mouse antibodies (mAK) can be isolated from the fluid of the ascites (and from the blood serum and the milk). The advantage of this method must above all be seen in the fact that the resulting amount of mAK Is considerably greater and that thus the efficiency of the inventive method can significantly be enhanced in comparison with former methods in which mice were exclusively used.

FIGS. 5A and 5B diagrammatically show a method of producing human monoclonal antibodies 13. Mice of the Balb-C inbred strain and a human individual serve, by way of example, as donors.

The method is first characterized in that two different tolerances must be induced in two different animal organisms. First of all, a mouse/man tolerance is induced with the aid of animal stem cells 4 of a donor 10 and human stem cells 1′ in an embryo 5 of an animal organism 6. After birth one obtains an organism 12 which exhibits double tolerance (mouse/man). (FIG. 5B).

In accordance with the above-explained principles a further animal 14 is produced with a human tolerance according to the illustration of FIG. 5A. To this end a human stem cell 1 is injected into an embryo 2 of another carrier animal 4. As has been stated, this results in the second animal 14 exhibiting human tolerance.

In the next step of the method, a suitable antigen 7 is injected into the animal 14 with a human tolerance, depending on the monoclonal antibodies to be produced. The human amount of the immune system of the animal 14 is thereby stimulated to form human antibody-producing B cells 8. These B cells 8 are taken from the animal 14 and isolated and fused with tumor cells 9 (myeloma cells) of the donor 10. This results in (man/mouse) hybridoma cells 11 which are injected into the animal 12 having a double tolerance (man/mouse tolerance). On account of its tolerance, this animal 12 does not reject the injected hybridoma cells 11, but rather forms an ascites 15 containing human monoclonal antibodies, a blood serum containing said antibodies or also milk containing said antibodies when a cow is, for instance, used as the animal.

The monoclonal antibodies 13 can now be isolated in the known manner from the respective fluid, e.g. the ascites 15 shown in FIG. 5B.

As for the above explanations, in particular the method according to FIGS. 5A and 5B, the following should additionally be noted:

Since in the above explanation regarding the embodiment according to FIGS. 5A, 5B, mice of the inbred strain Balb-C wore used as animal donors, the cells of the original donor 10 (see FIG. 5B) cannot be distinguished from the cells of the other Balb-C mice, since all are “quasi” genetically and phenotypically identical. This is the result of intensive inbreeding. During fusion of the human B cells 8 with the animal tumor cells 9, which also derive from Balb-C mice, it is thus enough that these cells 9 derive from the so-called Balb-C mice.

As has been explained above, the above-mentioned hybridoma cells are cells which are produced by cell fusion. In the case explained with reference to FIG. 5A, a B cell (lymphocyte subtype) is preferably fused with a myeloma cell.

Furthermore, it should be taken into account for explanatory purposes that a tumor is a clump of cells having the following characteristics:

a) uncontrolled cell growth;

b) uncontrolled cell migration (metastases),

c) “immortality” (or better: “longevity”);

d) partial disappearance of original cell characteristics in favor of new ones; and

e) all cells originate from a single degenerated cell (clonality).

A myeloma is a tumor of a B cell which is equipped with said characteristics (a-e).

Furthermore, to understand the present invention in an improved manner, it should be noted that a B cell produces a single antibody. Many B cells produce many different antibodies (=immunoglobulins are the fraction of proteins in plasma electrophoresis that contain all antibodies).

Since all tumor cells derive per definition from one cell, they only produce one antibody (M gradient in the plasma electrophoresis of such patients).

These characteristics are transferred to a normal donor B cell during fusion. Therefore, one talks about monoclonal antibodies (mAk). “Mono” means here “of one type” and “clonal” “derived from one cell”.

The tumor or myeloma cell 9 which has here been mentioned in connection with the explanation of FIG. 5A may e.g. be derived from the myeloma cell line XG3-AG8 of Balb-C mice, which is commercially available. Such myeloma cells can be used within the scope of the present invention as fusion partners.

For a supplementary disclosure and explanation, reference is made to the table shown on the next page and containing definitions of terms.

Furthermore, for a supplementary disclosure it should be added that after injection of the antigen 7 (see FIG. 5A) and fusion with the tumor cell and after growth carried out in the laboratory those cells are determined by testing that contain the antibodies stimulated by the specific antigen. A special advantage of the method according to the invention must here be seen in the fact that the number of cells which produce the corresponding specific antibodies is considerably increased by stimulation through a special antigen. The testing methods for determining the respective, specifically desired hybridoma cells are known per se.

### DEFINITION OF TERMS

| Term | Abbreviation | Definition |
|---|---|---|
| Antigen | Ag | Molecules which react with antibodies. |
| | | Their name results from their ability to |
| | | produce antibodies. However, since some |
| | | antigens cannot stimulate antibody pro- |
| | | duction by themselves, only those anti- |
| | | gens that can induce antibody production |
| | | are designated as immunogens. |
| Antibody | Ak/Ab | Immune bodies; reaction products of the |
| | | body cells to the stimuli of antigens |
| | | against which they are specifically di- |
| | | rected. Plasma proteins which specifi- |
| | | cally bind to molecules known as anti- |
| | | gens. Antibody molecules are produced |
| | | as a response to the immunization with |
| | | an antigen. They are the specific mole- |
| | | cules of the human immune reaction and |
| | | bind and neutralize phatogens or prepare |
| | | the same for absorption and destruction |
| | | by phargocytes. The antibody molecule |
| | | has a unique structure which enables the |
| | | molecule to bind to specific antigens. All |
| | | of the antibodies, however, have the same |
| | | overall structure and are designated, on |
| | | the whole, as immunoglobulins. |
| Monoclonal | mAK/mAB | Antibodies which are produced by a single |
| antibody | | nucleus of B lymphocytes. Monoclonal |
| | | antibodies are normally produced through |
| | | the production of hybrid antibody-forming |
| | | cells from the fusion of myeloma cells |
| | | with immune spleen cells. |
| B cells | — | B cells or B lymphocytes constitute one of |
| | | two main classes of lymphocytes. The an- |
| | | tigen receptor on B cells, which is part- |
| | | ly designated as a B cell receptor, is a |
| | | cell-surface immunoglobulin molecule. |
| | | Upon activation by an antigen B cells di- |
| | | fferentiate into cell-producing anti- |
| | | bodies of the same specifity as that of |
| | | the initializing receptor. |
| Balb-C | Balb-C | Specific inbred mouse strain. |
| mouse | | |
| Hemato- | — | Formation of the cellular elements of |
| poiesis | | blood, including the red blood cells, the |
| | | leukocytes and the blood patelets. |
| Hybridoma | — | Cell resulting from the fusion of a specific |
| | | antibody-producing B cell with a mye- |
| | | loma cell. |
| Myeloma | — | Tumor cell with B cell origin. |
| cell | | |
| Stem cells | — | Basophilic cells as the first stage of |
| | | leukocyte development. |
| Stem cells, | | Cells capable of self-renewal and differen- |
| hematologi- | | tiation, used in the clinical field for |
| cal | | hematopoietic re-constitution after irradia- |
| | | tion or chemotherapy (bone marrow trans- |
| | | plantation). |
| Tolerance | — | State of specific immunological non- |
| | | reactivity after contact with a normally |
| | | antigenic stimulus (in the case of a normal |
| | | reaction to other antigens). |

1. A method for using an animal organism
a. for producing monoclonal antibodies and/or
b. for producing immunoglobulins and blood components and/or
c. for producing polycgonal antibodies (antisera),
wherein the step of transplanting a human or animal stem cell into an inseminated ovum of the animal organism is carried out for producing an intact human or animal hematopoiesis.

2. The method according to claim 1, characterized in that the transplantation is carried out within the first third of fetal development.

3. The method according to claim 1 or 2, characterized in that the following cells are used as stem cells:
hematopoietic stem cells from bone marrow, umbilical cord blood, fetal liver or spleen and circulating blood stem cells in the peripheral blood circulation;
embryonal stem cells;
any kind of call culture (such as cell hybridomas) and tissue.

4. The method according to any one of claims 1 to 3, characterized in that cells from the donors man and/or mammals, such as in particular mouse, chicken, horse and ruminants and/or birds, such as in particular chicken, and/or cell cultures and hybridomas are particularly used as donor stem cells.

5. The method according to any one of claims 1 to 4, characterized in that particularly mammals, such as sheep, goat, ruminant (cow), horse or birds, in particular chicken, are used as animal organisms as recipients of the stem cells.

6. The method according to any one of claims 1 to 5, characterized in that transplantation is carried out in vitro.

7. The method according to any one of claims 1 to 5, characterized in that transplantation is carried out as an In utero transplantation.

8. The method according to any one of claims 1 to 5, characterized in that transplantation is carried out outside the maternal body.

9. The method according to any one of claims 1 to 8, characterized in that after birth of the animal organism, which has received the stem cells, the tolerant state is confirmed by molecular biological methods or by means of FACS methods.

10. A method for producing human monoclonal antibodies (13), comprising the following steps,
a. transplanting a human stem cell (1) of a specific human donor into an embryo (2) of a first animal organism (3);
b. transplanting an animal stem cell (4) of a donor (10) and a human stem cell (1′) of the specific human donor into an embryo (5) of a second animal organism (6);
c. injecting an antigen (7) for immunostimulating the first tolerant animal organism (14) born after transplantation according to step (a);
d. isolating human antibody-producing B cells (8) from the first tolerant organism (14);
e. fusing the B cells (8) with tumor cells (9), preferably myeloma cells, of an organism (10) donating the stem cells (4) according to step (b), for producing hybridoma cells;
f. injecting the hybridoma cells (11) into the second double-tolerant animal organism (12) born after transplantation according to step b.;
g. isolating the monoclonal human antibodies (13) from the resulting ascites (15), the resulting blood serum, the produced milk or the laid eggs of the second double-tolerant animal organism (12).

11. The method according to claim 10, characterized in that mammals, such as sheep, cows or horses, or birds, such as chicken, are used as the first and second animal organisms (3 and 6, respectively).

12. The method according to claim 10 or 11, characterized in that a mouse of the inbred strain Balb C is used as the donor (10).

13. The method according to any one of claims 10 to 12, characterized in that the tolerant state is checked after birth of the first and second tolerant animals (12, 14).

14. The method according to any one of claims 10 to 13, characterized in that use is made of antigens (7) from pathogens of infectious diseases, in particular malaria, TBC, rabies, measles, tetanus, FSME, herpes and HIV or antigens against tumors or blood group antigens (rhesus factor D) or histocompatibility antigens (HLA antigens).

15. A method for producing mouse-monoclonal antibodies, comprising the following steps:
a. transplanting a stem cell (FLZ) of a mouse donor animal (M) into an embryo (E) of an animal organism (T);
b. injecting hybridoma cells (HC) of the mouse-donor animal (M) into the mouse-tolerant animal organism (C) born after transplantation according to step a.; and
c. isolating mouse-monoclonal antibodies (mAb) from the resulting ascites (ASC), the resulting blood serum, the produced milk or the laid eggs of the mouse-tolerant animal (C).

16. The method according to claim 15, characterized in that sheep or cows or chicken are used as animal organisms.

17. The method according to claim 15 or 16, characterized in that a mouse of the inbred strain Balb-C is used as the donor.

18. The method according to any one of claims 15 to 17, characterized In that after birth of the tolerant organism the tolerant state is checked.

19. The method according to any one of claims 15 to 18, characterized in that use is made of antigens (7) from pathogens of infectious diseases, in particular malaria, TBC, rabies, measles, tetanus, FSME, herpes and HIV or antigens against tumors or blood group antigens (rhesus factor D) or histocompatibility antigens (HLA antigens).

20. A bioreactor for producing monoclonal antibodies, comprising
at least one part of a life-sustained animal body having a functioning blood circulation; and
at least one functioning organ of the animal body in which after injection of antibody-producing hybridoma cells of a donor monoclonal antibodies can be produced or after injection of antigens antisera can be produced or are producible in the blood products,
wherein the animal body is tolerant to the donor of the cells.

21. The bioreactor according to claim 20, characterized in that the animal body is kept alive with technical equipment.

22. The bioreactor according to claim 20, characterized in that the part of the animal body is part of a mammal or bird naturally kept alive.

23. The bioreactor according to any one of claims 20 to 22, characterized in that the donor is a mammal, e.g. a mouse, a ruminant, a horse or a goat, and that the animal body is tolerant to the donor.

24. The bioreactor according to any one of claims 20 to 22, characterized in that the animal body exhibits double tolerance.

25. The bioreactor according to claim 24, characterized in that the double tolerance is a mouse/man tolerance.

26. The bioreactor according to claims 20, 24 or 25, characterized in that the hybridoma cell (11) is a fusion cell from a mouse-derived tumor cell (9) and a B cell (8) producing human antibodies or mouse antibodies.
